(19) United States
(12) Patent Application Publication
(10) Pub. No.: US 2021/0069457 A1
(43) Pub. Date: Mar. 11, 2021

(54) Pre and Post Anesthetic Cooling Device and Method

(71) Applicant: Jennifer Pilby Gomez, Carlsbad, CA (US)

(72) Inventors: Jennifer Pilby Gomez, Alpine, CA (US); Joshua Morrison, San Diego, CA (US)

(21) Appl. No.: 17/020,762

(22) Filed: Sep. 14, 2020

Related U.S. Application Data
(63) Continuation-in-part of application No. 16/242,823, filed on Jan. 8, 2019.
(60) Provisional application No. 62/900,149, filed on Sep. 13, 2019.
(60) Provisional application No. 62/614,557, filed on Jan. 8, 2018.

(51) Int. Cl. A61M 19/00; A61F 7/00

(52) U.S. Cl. CPC A61M 19/00; A61F 7/0085; A61F 2007/0096; A61F 2007/0076; A61F 2007/0087; A61F 2007/0056

(57) ABSTRACT

The present disclosure comprises a pre and post anesthetic cooling apparatus, system, and method for using thereof. In an embodiment, an anesthetic cooling device comprises a wand comprising a proximal end and a distal end opposite the proximal end, a thermoelectric module disposed within the wand having a cold side and a hot side, a TEM heat exchanger disposed within wand and connected to the hot side of the thermoelectric module, wherein a coolant circulates into the TEM heat exchanger through a wand inlet and out of the TEM heat exchanger through a wand outlet, a tip located at the distal end of the wand and connected to the TEM heat exchanger through a thermal conductor; wherein thermal energy is transferred from the tip to the coolant via the thermal conductor and the TEM heat exchanger.

### CROSS-REFERENCE TO RELATED APPLICATIONS

The present application claims priority to U.S. Provisional Patent Application No. 62/900,149 filed on Sep. 13, 2019 and entitled, “Pre and Post Anesthetic Cooling Device and Method,” and U.S. patent application Ser. No. 16/242,823 filed on Jan. 1, 2019 and entitled, “Recirculating Anesthetic Cooling Apparatus and Method,” the entire disclosures of which is incorporated by reference.

### BACKGROUND OF THE INVENTION

### 1. Field of Invention

The present disclosure relates to an anesthetic cooling device and, more specifically, a device or device accessory to deliver anesthesia through cooling of biological tissue, such as the epidermis, mucocutaneous membranes, teeth, and the oral mucosa, thereby anesthetizing tissue by inhibiting nerve conduction through cooling providing a pain free injection.

### 2. Description of Related Art

There is a constant search for ways to avoid the invasive, and often painful, nature of the injection, and to find more comfortable and pleasant means for anesthesia before medical and dental procedures. Currently used before injections are topical anesthetics such as benzocaine. Benzocaine takes at least two minutes to anesthetize tissues in the mouth and five minutes or longer to anesthetize the skin. There have been reports of serious life-threatening adverse effects (e.g., seizures, coma, irregular heartbeat, respiratory depression) with over-application of topical anesthetics or when applying topical products that contain high concentrations of benzocaine to the skin. Over application of oral anesthetics such as benzocaine can increase the risk of pulmonary aspiration by relaxing the gag-reflex and allowing regurgitated stomach contents or oral secretions to enter a patient's airway. The topical use of higher concentration (for example, 14-20%) benzocaine products applied to the mouth or mucous membranes has been found to be a cause of methemoglobinemia, a disorder in which the amount of oxygen carried by the blood is greatly reduced. This side effect is most common in children under two years of age. As a result, the U.S. Food and Drug Administration (“FDA”) has indicated that benzocaine products should not be used in children under two years of age, unless directed and supervised by a healthcare professional. Symptoms of methemoglobinemia usually occur within minutes of applying benzocaine and can occur upon the first-time use or after additional use. Benzocaine may cause allergic reactions such as contact dermatitis and in rare events anaphylaxis.

Within the oral cavity there is keratinized tissue in the hard palate of the mouth. Benzocaine is ineffective especially through keratinized tissue of the hard palate. Topical gel cannot penetrate through this layer and therefore is ineffective at numbing keratinized tissue such as the roof of the mouth. As in the mouth, the skin has keratinized tissue, called the stratum corneum, through which topical 20% benzocaine is not easily absorbed.

Keratins are a diverse group of structural proteins that form intermediate filament networks and provide structural integrity to keratinized epithelial cells. The surface cells (outermost layer) of the keratinized epithelia are dead cells. Typically, protoplasm in the surface cells of the keratinized epithelium is replaced by keratin to provide better protection against abrasions and is impervious to water.

Many dental patients across the United States received local anesthetic injections every year. Currently there are about 200,000 dentists that practice in the United States. The American Dental Association last survey of dental services in 2005-2006 reported about 73.8 million procedures that required injections. These estimates didn't even include procedures requiring the removal of cavities and with the survey being over ten years, these numbers are grossly underestimated.

In general, a thermoelectric cooler comprises two sides, and when the DC electricity flows through the device (i.e., the device is energized), it brings heat from one side to the other, so that one side becomes cooler while the other side becomes hotter.

A thermoelectric (“TE”) module, also called a thermoelectric cooler or Peltier cooler, is a semiconductor-based electronic component that functions as a small heat pump, moving heat from one side of the device to the other. By applying a low voltage DC power to a TE module, heat will be moved through the module from one side to the other. One module face, therefore, will be cooled while the opposite face is simultaneously heated. Consequently, a thermoelectric module may be used for cooling thereby making it highly suitable for precise temperature control applications. A practical thermoelectric module generally consists of two or more elements of n and p-type doped semiconductor material that are connected electrically in series and thermally in parallel. These thermoelectric elements and their electrical interconnects typically are mounted between two ceramic substrates. The substrates hold the overall structure together mechanically and electrically insulate the individual elements from one another and from external mounting surfaces.

Both N-type and P-type Bismuth Telluride thermoelectric materials are used in a thermoelectric cooler. This arrangement causes heat to move through the cooler in one direction only while the electrical current moves back and forth alternately between the top and bottom substrates through each N and P element. N-type material is doped so that it will have an excess of electrons (more electrons than needed to complete a perfect molecular lattice structure) and P-type material is doped so that it will have a deficiency of electrons (fewer electrons than are necessary to complete a perfect lattice structure). The extra electrons in the N material and the “holes” resulting from the deficiency of electrons in the P material are the carriers which move the heat energy through the thermoelectric material. Most thermoelectric cooling modules are fabricated with an equal number of N-type and P-type elements where one N and P element pair form a thermoelectric “couple.”

Cooling capacity (heat actively pumped through the thermoelectric module) is proportional to the magnitude of the applied DC electric current and the thermal conditions on each side of the module. By varying the input current from zero to maximum, it is possible to regulate the heat flow and control the surface temperature.

A heat pipe is a heat-transfer device that combines the principles of both thermal conductivity and phase transition to effectively transfer heat the hot interface of a heat pipe a liquid in contact with a thermally conductive solid surface turns into a vapor by absorbing heat from that surface. The vapor then travels along the heat pipe to the cold interface and condenses back into a liquid, releasing the latent heat. The liquid then returns to the hot interface through either capillary action, centrifugal force, or gravity, and the cycle repeats. Due to the very high heat transfer coefficients for boiling and condensation, heat pipes are highly effective thermal conductors. They can be made with ammonia and methanol inside the pipe to provide heat transport in below freezing temperatures.

Examples of thermoelectric cryoprobes include U.S. Pat. No. 5,207,674 to Hamilton (hereinafter, “Hamilton”), U.S. Patent Application Publication No. 20160242956 to Pilby Gomez (hereinafter, “Pilby Gomez”), U.S. Pat. No. 3,618,590 to Frank, et al. (hereinafter, “Frank”), U.S. Pat. No. 4,483,341 to Witteles (hereinafter, “Witteles”) and U.S. Pat. No. 5,209,227 Deutsch (hereinafter, “Deutsch”).

Deutsch discloses a heating device that uses a thermally conductive head with a fan to dissipate heat generated by the Peltier effect. Deutsch fails to be effective because miniaturizing the fan to be small enough to fit inside the crevices of the mouth would greatly reduce air flow, therefore ineffectively removing heat out of the system. Also, the device must be covered in a protective sheath for hygienic purposes, this wouldn't allow any air to flow making the fan ineffective.

Frank discloses a thermoelectric probe for applying heat or cold to a localized area of the body. The thermoelectric module is located in the head mounted in thermal conductive contact with a heat transfer unit also in the head. The heat transfer unit is composed of a honeycomb assembly of tubes leading out through the body to an external air vacuum pump. However, Frank is deficient because it makes it impossible for miniaturization of the head due to the necessary air flow needed to remove the heat coming off of the thermoelectric plate. For all dental applications, the head needs to me miniaturized to fit into the small crevices of the mouth.

Pilby Gomez discloses a thermoelectric plate in the head in thermal conductive contact with a heat pipe that runs along the length of the device. In the body of the device the same heat pipe is in thermal conductive contact with the heat sink, a solid copper metal slug. In another embodiment, the thermoelectric plate is in the body with the cold side thermally attached to the heat pipe and the hot side thermally attached to the copper heat slug. However, Pilby Gomez is deficient because the copper heat slug has a finite heat capacity and must be cooled back down to be able to continue working. The copper heat slug can be cooled back down to temperature through time, allowing the heat to dissipate away or it needs to be placed directly on a cold block to quickly get it back down to the desired temperature so it can function as a heat sink for the next application.

Hamilton discloses an electronic cryogenic surgical probe that uses multiple freeze-thaw cycles to destroy tissue and thereby treating benign and malignant tumors of the skin and mucous membranes. Hamilton is deficient because it does not disclose how the fluid moves through the device.

Witteles discloses an implantable hypothermia instrument for the in-situ treatment of oncological disorders. However, Witteles is deficient because it is meant to be implantable having three layers composed of TE plates, thermomagnetics, and Ettingshausen third cooling section connected in parallel. This does not have efficient cooling capacity to remove excess heat produced by the hot side of a thermoelectric module.

Therefore, what is needed is a more effective way of removing excess heat from the distal head end portion, in direct contact with patient's warm tissues, of the wand of the device through its body and out into the environment. The pathway and components utilized need to be addressed so the clinician can continuously use the anesthetic cooling device for many injection sites. This need has heretofore remained unsatisfied.

### SUMMARY OF THE INVENTION

The present disclosure overcomes these and other deficiencies of the prior art by providing an effective heat removal pathway and the components involved.

The present device improves patient comfort by providing fast, effective, anesthesia. The cooling device is much faster than topical anesthetics by anesthetizing the tissues in 20-30 seconds versus 2-5 minutes. This time difference is important to practitioners in a busy practice and they would prefer to have fast, effective pain control, especially for use on phobic patients.

Nerve cooling using thermoelectric plates can achieve a conduction block in the peripheral nerves that will allow for a pain free injection. Complete nerve conduction block occurs for temperatures below 0° C., quickly reversible, not messy, and has non nerve fiber selectivity. Ice is effective but drawbacks include a watery mess, and in the mouth causes tooth pain if cold water touches the tooth. Additional obstacles of using ice are the additional steps of taking it out of the freezer, and preparation to make sure its sanitary for patient use. A frozen cotton tip applicator melts too fast in the mouth making it difficult to maintain the target temperature for numbing.

Transient nerve cooling block of the peripheral nerves system is achieved by the presently disclosed approach using a thermoelectric cooling device. The thermoelectric cooling device allows for temperature controlled cooling of the epidermis, mucosa, hard palate, and gingiva to alleviate pain associated with injections for dental and medical treatment. In some embodiments, the present invention utilizes Peltier junctions to cool the patient's tissues, resulting in a numbing the nerves before penetration of needle or other operation. Many people avoid going to a dentist because they are scared of injections—the present invention helps alleviate this problem.

In some embodiments, the present invention can be applied to oral tissues. In other embodiments, the present invention is applied to tissues other than oral. The ability to deliver local anesthetics into nerves to numb the teeth and tissues is vital to dentistry. Unfortunately, in order to do this, it requires a painful injection. Currently, to give an intra oral injection, the dentist provides surface anesthesia by applying a small amount of 20% benzocaine gel using a cotton applicator to the mucosa to try to achieve topical anesthesia of 1-2 mm depth. After waiting 2 minutes, the surface tissue may or may not be anesthetized. Next, the dentist may use a syringe with lidocaine by inserting the needle tip into the area that the 20% benzocaine was applied, advancing the needle into the tissues until the tip has reached the target site. Once the tip has reached the target site, then the dentist expels the local anesthetic, bathing the nerve leading to the teeth and tissue needing to be numbed up. Once the target nerve is completely anesthetized the dentist can start the procedure.

The present invention is directed to a novel anesthetic cooling device and method for transient nerve cooling block of the peripheral nervous system. The thermoelectric cooling device of the present invention allows for temperature controlled cooling of the skin/epidermis or mucous membrane/mucosa, in order to alleviate pain associated with medical treatment such as injections, curettage, and skin ablation applied to the human body. The invention can also be used for pulp vitality testing, i.e., used as a clinical and diagnostic aid for dentistry to help establish the health of the dental pulp within the pulp chamber and root canals of a tooth. In such an embodiment, the cold end (the tip) of the wand touches the tooth to see if there is a response or not. This test informs the practitioner if the tooth is vital or not. The wand body of the cooling device comprises a proximal gripping end (i.e., handle) connected to a distal head section by a neck section.

The user (i.e., a clinician) first numbs the target tissue by placing the cooling tip in the distal head section of the thermoelectric cooling device against the tissue. The cool side covered with a hygiene barrier sleeve contacts the tissue for 60 seconds or less until the tissue reaches 0° C. or less. Then, the clinician removes the thermoelectric device and inserts the hypodermic needle into the target tissue, thereby allowing for a pain-free experience for the patient.

Although the embodiments discussed herein are discussed in the context of tissues located in and around a patient's mouth, the present invention can be used with tissues located anywhere on a patient's body without departing from the contemplated embodiments. Additionally, the present invention can be used on human patients as well any animal's without departing from the contemplated embodiments.

In contrast to this prior art, the present invention provides a novel efficient heat transfer path, removing thermal energy (i.e., heat) from the tissue into the distal end of the wand, through the wand's body using a specialized thermoelectric module (“TEM”) heat exchanger in the wand of the anesthetic cooling device. The parts and mechanism of how heat from the tissue is removed by the wand of the cooling device, specifically from the hot side of the thermoelectric plate to outside of the wand and into the ambient environment. The wand of the anesthetic cooling device accomplishes this functionality. For example, the described process may be accomplished by using a novel specialized heat sink or TEM heat exchanger with channels coupled directly or indirectly through a heat spreader to a heat pipe/vapor chamber or solid metal thermal conductor. Water, air, and other fluids can be used as the coolant flowing through the metal looped block. The block may rely on “looping” designs or, alternatively, other shapes/patterns, e.g., zig zags or right angles, starting at the inlet and terminating at the outlet. Four configurations are contemplated.

In some embodiments of the present invention, a thermoelectric cooling module (also referred to as a “thermoelectric cooling plate” or “thermoelectric module”) is utilized to cool the tip located within the wand. A thermoelectric cooling plate generally uses the Peltier effect to create a heat flux at a junction of two different types of materials. Such a thermoelectric cooler is a solid-state active heat pump which transfers heat from one side of the device to the other using electrical energy. The side of the thermoelectric plate to which heat is transferred is referred to as the “hot side.” Conversely, the side of the thermoelectric plate from which the thermal energy is transferred is referred to as the “cold side.” In other words, when the thermoelectric plate is in use, the cold side is cold, and the hot side is hot. In some embodiments, a single thermoelectric plate may be employed. In other embodiments, a plurality of thermoelectric plates may be used.

In an embodiment, the hot side of the thermoelectric module in the distal head end are coupled to the distal end of a thermal conductor in the wand of the anesthetic cooling device. A heat spreader in the proximal end of the wand is connected on the opposite end of thermal conductor. The heat spreader is coupled to the internal heat sink or specialized TEM heat exchanger which is coupled to the inlet and outlet tubes.

In another embodiment, the hot side of the thermoelectric module in the distal head end are coupled to the distal end of the thermal conductor in the wand of the anesthetic cooling device. The internal heat sink or specialized TEM heat exchanger in the handle is coupled to the other end of the heat pipe or solid metal thermal conductor. The TEM heat exchanger is coupled to the inlet and outlet tubes.

In another embodiment, the cold side of the thermoelectric modules is coupled to the heat spreader on the proximal end of the heat pipe/vapor chamber or solid metal thermal conductor in the handle of the wand of the anesthetic cooling device. The hot side of the thermoelectric plates is coupled to the internal specialized heat sink or TEM heat exchanger which is coupled to the inlet and outlet tubes.

In another embodiment, the cold side of the thermoelectric modules are coupled directly to the proximal end of the thermal conductor in the handle of the wand of the anesthetic cooling device. The hot side of the thermoelectric plates in the handle are coupled to the internal specialized heat sink or TEM heat exchanger which is coupled to the inlet and outlet tubes.

The special shaped spreader and TEM heat exchanger are internal passive heat exchangers that transfer the heat generated from the hot side of the thermoelectric module to a coolant such as air or liquid, where the heat is dissipated away from the device, thereby allowing regulation of the device's temperature at optimal levels.

Thermal resistance refers to a material's ability to resist heat transfer. As a practical matter, the lower a material's thermal resistance, the more efficient that material is at dissipating heat. In another embodiment of the present invention, the heat flow between the thermoelectric module and the ambient environment is modeled as a series of resistances to heat flow. That is, there is a resistance from the hot side of the thermoelectric plate to the heat pipe, heat pipe to the heat sink exchanger, and from the heat sink exchanger to the circulating coolant. In another embodiment, there is a resistance from the hot side of the thermoelectric plate to the internal heat sink exchanger or TEM heat exchanger, and from the heat sink exchanger or TEM heat exchanger to the forced air or water. In some embodiments, to reduce the thermal resistance and increase the conductivity of the system, the cooling device uses a spreader, heat sink exchanger, or TEM heat exchanger, and thermo-conductive materials such as, for example, copper, aluminum, thermal bond paste, and/or grease.

In another embodiment, conduction occurs from the hot side of the thermoelectric module when the hot side of the thermoelectric module contacts the heat pipe or solid metal thermal conductor by thermal epoxy or thermal grease. In such an embodiment, at the point where the two components meet, the faster moving molecules from the hot side of the thermoelectric plate interact with the slower moving molecules of the evaporator end of the heat pipe or into the slower end of the solid metal thermal conductor. The fast moving molecules from the heat pipe or solid metal thermal conductor interact with the heat spreader, raising the temperature of the spreader. The molecules within the spreader move faster, spreading the thermal energy to the heat sink or specialized TEM heat exchanger, and/or the liquid coolant traversing within the specialized looped coolant block transferring the energy outside of the device.

In another embodiment, heat conduction occurs from patient tissue to the heat pipe or solid metal thermal conductor, then from the cold side of the thermoelectric module to the hot side of the thermoelectric module. At the point where the two components meet, the faster moving molecules from the hot side of the thermoelectric plate interact with the slower moving molecules of the internal heat sink or TEM heat exchanger. The molecules contained within the hot side of the thermoelectric module move faster, spreading the thermal energy to the specialized TEM heat exchanger, then liquid coolant moving through the TEM heat exchanger transfers the energy outside of the wand and into a recirculating chiller that transfers the thermal energy to the ambient environment.

Convective heat transfer, i.e., convection, is the transfer of thermal energy from one place to another by the movement of fluids. Convection is usually the dominant form of heat transfer in liquids and gases. Although often discussed as a distinct method of heat transfer, convective heat transfer involves the combined processes of conduction which is heat diffusion, and advection, which is heat transfer by bulk fluid flow. In the cooling device's wand, the heat transfer from the heat sink/fluid loop metal block to the ambient environment occurs by forced convection from the incoming air through the inlet, conduction concurrently occurs through the metal of the heat sink, and out through the outlet, the remaining heat escaping through radiation. The incoming fluid flow can be created from an external air or water pump.

Another parameter that concerns the thermal conductivity of the specialized heat sink is spreading thermal resistance. Spreading thermal resistance occurs when thermal energy is transferred from a small area such as from the thermoelectric plate to a larger area, the heat sink, having finite thermal conductivity. In some instances, heat energy will not distribute uniformly through the heat sink without the help of a heat spreader. The spreading resistance phenomenon is shown by how the thermal energy travels from the heat source location and causes a large temperature gradient between the heat source and the edges of the heat sink. This means that some areas are at a lower temperature than if the heat source were uniform across the base of the heat sink. This non-uniformity increases the heat sink's effective thermal resistance. To overcome thermal resistance in the base of the heat sink, the base thickness (i.e., thermal mass) can be increased using a conductive plate, a spreader to disburse the thermal energy (i.e., heat), preventing a large temperature gradient.

The specialized heat sink may be made of metal, which serves as the thermal conductor, carrying heat away from the thermoelectric plates. Another way the thermal resistance in the base of a heat sink is decreased is to employ materials with higher thermal conductivities such as, for example, copper and aluminum.

The specialized heat sink/fluid loop metal block is designed to be in contact with the cooling medium within it—the coolant. The coolant can be any suitable fluid such as, for example, water, alcohol, or air. In some embodiments, additives can be used with the coolant to increase its efficiency. For example, water may be used as a coolant with additives that increase the water's thermal capacity. Additives may also be used with the coolant that assist in other aspects of the present invention. For example, an additive may be added to water-based coolant that mitigates/prevents rust or other corrosion within the various components of the present invention.

In another embodiment, thermal adhesive or thermal grease may be employed to improve the heat sink's performance by filling air gaps between the heat sink and the hot side of the thermoelectric module in the wand of the cooling device. Additionally, employing materials with high thermal coefficients will also increase the present invention's efficiency. For example, in some embodiments, the heat sink may be made from copper or aluminum. The heat pipe or solid metal thermal conductor in the body of the wand can be made out of copper. This allows for the solid metal conductor to efficiently dissipate and transfer thermal energy due to copper's desirable thermal properties.

Copper may be used because it has many desirable properties such as being thermally efficient and durable. First and foremost, copper is an excellent conductor of heat. This means that copper's high thermal conductivity allows heat to pass through it quickly. Aluminum has a lower thermal conductivity than copper but still is very thermally conductive and lightweight, which is important for the clinician during cooling device operation.

A pump is used to circulate the coolant throughout the present invention. For example, in an embodiment where air or water is used as the coolant, an air or water pump moves air or water or other fluid into the wand through the inlet, across the fluid loop metal block and out of the wand through the outlet. In an example where air is used as the coolant, the cool air is drawn into the wand portion of the cooling device through the inlet and is blown through the fluid loop metal block, removing the heat from inside the wand heating the air, and expelled out of the outlet.

In an example where water or other liquid coolant is used, a water block, coolant block, or TEM heat exchanger is employed to cool the hot side of the thermoelectric plate and is the water-cooling equivalent of a heatsink. In such an embodiment, the heatsink comprises a base, which is the area that makes contact with the thermoelectric plate or heat spreader being cooled, and usually comprises metals and other materials with high thermal conductivity such as aluminum or copper. In such an embodiment, the cooling apparatus further comprises a top portion that ensures the liquid coolant is contained safely inside the TEM heat exchanger and has connections that allow hosing to connect it with the closed liquid cooling loop from the external coolant chiller. The top can be made of the same metal as the base, transparent Perspex, Delrin, Nylon, or HDPE.

In some embodiments, the base, top, and mid-plate(s) are sealed together to form a block with a path for liquid coolant to flow through. The TEM heat exchanger can also be made as a solid metal block having channels of variable sizes and shapes therethrough. The ends of the liquid loop block path have inlet and outlet connectors for tubing connected to the external chiller. A TEM heat exchanger is better at dissipating heat than an air-cooled heatsink due to water's higher specific heat capacity and thermal conductivity. The water or other liquid coolant from the wand is pumped through to a recirculating chiller. The recirculating chilled liquid coolant passes through the looped liquid metal block inside the wand. The thermal energy from the hot side of the thermoelectric module is transferred to the coolant, resulting in the coolant's temperature being raised. The warmed coolant is returned to the chiller for re-cooling. In such an embodiment, the described coolant loop has a water pump and reservoir that completes the closed loop system. Thermoelectric modules can also be used to cool the liquid in the external recirculating chiller.

In another embodiment of the present disclosure, a closed loop system using a radiator is utilized. A radiator closed loop with a liquid coolant (for example, water, propylene glycol, or a mix of both) enters the TEM heat exchanger at approximately ambient air temperatures. The exiting warm liquid then passes through a fan cooled radiator of sufficient size to cool the liquid; in some embodiments, the coolant is cooled to ambient air temperature. The coolant is then sent back through the coolant block.

In some embodiments, tap water can be used as a coolant to circulate through the TEM heat exchanger. This water (coolant) is usually cooler than the ambient air temperature. The warmer exiting water (coolant) can be deposited to a drain or, alternatively, be re-used in a number of ways. In dental applications, the tubing that connects to the inlet of the wand can also be connected to the dental operatory quick release water and air connections and the outlet tubing can go into the chair or wall suction.

In some embodiments, the cooling loop hardware comprises corrosion resistant alloys, such as copper alloys and stainless steels. Ethylene propylene diene monomer (“EPDM”) rubber may form the inner lining of all the hoses in the system. The chemistry of the cooling water must be properly maintained to avoid system disruption or shutdown due to any of the four common water-related problems of corrosion, microbiological growth, scale formation, and fouling.

Installation of a TEM heat exchanger is also similar to that of a heatsink, a thermal pad or thermal grease may be placed between the TEM heat exchanger and the hot side of the thermoelectric module being cooled to aid in heat conduction.

Using liquid coolant to remove thermal energy from the thermoelectric modules allows for precise temperature control. Using a TEM heat exchanger to dissipate heat from one component to another, and maintain the coolant's temperature throughout the loop and to prevent components from overheating. Circulating a coolant to the thermoelectric modules allows the tip of the anesthetic cooling device to achieve temperatures sufficient to achieve the desired effects, as described herein.

In some embodiments, the cooling device can have precise temperature control from 6° C. to −15° C. due to the use of the heat spreader and liquid loop block heat transfer efficiency. However, temperatures above 6° C. and below −15° C. can be used without departing from the contemplated embodiments.

In some embodiments of the present invention, an external coolant chiller with thermoelectric plates and a fluid reservoir is used. In such embodiments, the liquid coolant can be chilled using a thermoelectric plates. The external reservoir and thermoelectric plates are connected to a pump that circulates coolant from the external chiller to the wand of the cooling device. Coolant is circulated from the external chiller, through the inlet to the wand, and then from the outlet of the wand back to the external chiller, completing the closed circuit. This type of circuit, with an external reservoir containing thermoelectric plates allows for a higher capacity of heat removal from the wand of the anesthetic cooling device. Additional coolant may be stored in the reservoir and used to replenish any losses of coolant volume.

In some embodiments, the liquid reservoir comprises thermoelectric plates directly attached thereto for a compact, efficient design. In other embodiments, the liquid reservoir can be separate from the thermoelectric plates, with the thermoelectric plates as its own liquid chilling unit. The liquid chiller, reservoir, pump, thermoelectric controller, and power supply can all be housed in one external unit.

In some embodiments, an external coolant chiller is used to supply a plurality of wands. For example, in an embodiment where the anesthetic cooling device is used in a dental office, the external coolant chiller may be located in a room separate from the patient. In such an embodiment, hoses or tubes may be built into the facility such that coolant is circulate from the external coolant chiller to the wand. In such an embodiment, multiple wands may be located in different rooms, all of which may be operatively connected to the external coolant chiller located in a separate area of the dental facility. Such an embodiment allows a single external coolant chiller to be connected to a plurality of wands. Additionally, any noise, smell, vibration, or other unwanted phenomena generated by the external coolant chiller is isolated and kept away from the patient area.

In other embodiments, the present invention uses a solid cooling device without inlets or outlets. In such an embodiment of the anesthetic cooling device, the wand is made from solid conductive metal that has no inlet or outlets connected to an external unit to remove heat. In such an embodiment, the cooling device is cradled inside a cooling cradle at a specific temperature, for example, below 0° C. Heat is transferred from the solid metal inside the cooling device into the cooling cradle through conduction, cooling the metal inside the anesthetic cooling device to the desired temperature. The cooled metal is used to remove heat from the tissues by cooling and therefore numbing the tissues. In such an embodiment, the want may comprise an insulative material such as plastic or other polymer to surround the top or handle portion of the solid cooling wand for hand comfort and to help prevent the anesthetic cooling device from losing efficacy by being warmed by the ambient air.

In another exemplary embodiment, the anesthetic cooling device uses a cooling cradle. In such an embodiment, the cooling cradle provides primary or additional heat removal for the anesthetic cooling device. In an embodiment, the cooling cradle comprises a metal plate shaped for the anesthetic cooling device to fit into on top of a thermoelectric plate with its own heat sink comprising fins and/or a fan. The cooling cradle temperature can be set to a desired temperature, for example, below freezing. The cooling device fits inside or on top of the cooling cradle, providing contact between the cooling device and cooling cradle. This contact allows for conduction to occur from the cooling device to the cooling cradle, thereby bringing the temperature of the cooling device to the desired temperature (e.g., below freezing). The cooling cradle provides primary heat transfer from the metal body of the cooling device. The shape of the cooling cradle can be curved or flat depending on the size and shape of the anesthetic cooling device employed. In some embodiments, the cooling cradle comprises a top portion made from thermo-conductive material such as copper or aluminum or from thermo-insulative materials such as plastic or other polymers. In some embodiments, the top portion and bottom portion of the cooling cradle are configured such that the internal cavity is shaped to match the wand. In such an embodiment, the top portion and bottom portion hinge apart, allowing the user to open and close the cooling cradle.

In another exemplary embodiment of the present invention, removable cooling packs are employed to lower the temperature of the anesthetic cooling device. Such removable cooling packs may provide primary or additional heat removal. The packs are removed from an external freezer and placed inside the cooling device through an opening to provide primary or adjunctive heat removal. The frozen packs are located against the thermoelectric modules or heat sinks any embodiment.

In another exemplary embodiment of the present invention, an anesthetic cooling device comprises a wand comprising a proximal end and a distal end opposite the proximal end, a thermoelectric module disposed within the wand having a cold side and a hot side, a TEM heat exchanger disposed within wand and connected to the hot side of the thermoelectric module, wherein a coolant circulates into the TEM heat exchanger through a wand inlet and out of the TEM heat exchanger through a wand outlet, a tip located at the distal end of the wand and connected to the TEM heat exchanger through a thermal conductor; wherein thermal energy is transferred from the tip to the coolant via the thermal conductor and the TEM heat exchanger. In another exemplary embodiment, the present invention further comprises an external coolant chiller comprising an inlet, an outlet, a pump, a power source, a reservoir, and a second heat exchanger; wherein the pump circulates coolant from the wand outlet, through the second heat exchanger, and into the TEM heat exchanger via the wand inlet; and wherein the second heat exchanger lowers the temperature of the coolant. In another exemplary embodiment of the present invention, the external coolant chiller further comprises a second thermoelectric module. In another exemplary embodiment, the present invention further comprises a second thermoelectric module comprising a hot side and a cold side; wherein the cold side of the second thermoelectric module is connected to the hot side of the thermoelectric module. In another exemplary embodiment of the present invention, the tip reaches a temperature of below 0° C. In another exemplary embodiment of the present invention, the tip is interchangeable. In another exemplary embodiment, the present invention further comprises a temperature sensor. In another exemplary embodiment of the present invention, the TEM heat exchanger is connected to the hot side of the thermoelectric module via a heat spreader.

In another exemplary embodiment of the present invention, a method of anesthetic cooling comprising: contacting a patient's tissue with a tip located at the distal end of a wand; transferring thermal energy, using a thermoelectric module, from the tip to a TEM heat exchanger, wherein the TEM heat exchanger is connected to a hot side of the thermoelectric module; and circulating a coolant into the TEM heat exchanger through a wand inlet and out of the TEM heat exchanger through a wand outlet; wherein the thermal energy is transferred from the tip to the TEM heat exchanger through a thermal conductor, wherein the thermal conductor is connected to the tip and to a cold side of the thermoelectric module. In another exemplary embodiment, the present invention further comprises circulating the coolant from the wand outlet to an inlet of an external coolant chiller; reducing the temperature of the coolant using a second heat exchanger located within the external coolant chiller; and circulating the coolant to the wand inlet of the TEM heat exchanger; wherein the external coolant chiller comprises an outlet, a pump, a power source, and a reservoir. In another exemplary embodiment of the present invention, a second thermoelectric module is used to transfer the thermal energy from the tip to a TEM heat exchanger. In another exemplary embodiment of the present invention, the external coolant chiller further comprises a second thermoelectric module. In another exemplary embodiment of the present invention, the tip reaches a temperature of below 0° C. In another exemplary embodiment of the present invention, the tip is interchangeable. In another exemplary embodiment, the present invention further comprises the step of monitoring the temperature of the tip using a temperature sensor located within the wand. In another exemplary embodiment of the present invention, the TEM heat exchanger is connected to the hot side of the thermoelectric module via a heat spreader.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present disclosure, the objects and advantages thereof, reference is now made to the ensuing descriptions taken in connection with the accompanying drawings briefly described as follows:

FIG. 1 depicts a top perspective see through view of a thermoelectric anesthetic cooling device wand including the tip, the heat pipe/vapor chamber, or solid metal thermal conductor running along the length of the device, the thermoelectric plates in parallel, and specialized TEM heat exchanger, according to an exemplary embodiment of the present disclosure;

FIG. 2 depicts a see through view of a thermoelectric anesthetic cooling device wand including the tip, the heat pipe/vapor chamber, or solid metal thermal conductor running along the length of the device, the thermoelectric plates in parallel, and specialized TEM heat exchanger, according to an exemplary embodiment of the present disclosure;

FIG. 3 depicts a cross-sectional view of a thermoelectric anesthetic cooling device wand including the tip, the heat pipe/vapor chamber, or solid metal thermal conductor running along the length of the device, the thermoelectric plates in parallel, and specialized TEM heat exchanger, according to an exemplary embodiment of the present disclosure;

FIG. 4 depicts a side perspective view of a specialized TEM heat exchanger in the body of the wand, along with the channels, according to an exemplary embodiment of the present disclosure;

FIG. 5 depicts a top perspective view of a specialized TEM heat exchanger in the body of the wand, along with the channels, according to an exemplary embodiment of the present disclosure;

FIG. 6 depicts a see through perspective view of a specialized TEM heat exchanger in the body of the wand, along with the channels, according to an exemplary embodiment of the present disclosure;

FIG. 7 depicts a transverse perspective view of the front end of the specialized TEM heat exchanger in the body of the wand, TE module, and heat pipe/vapor chamber, or solid metal thermal conductor;

FIG. 8 depicts a top perspective view of the pathway through the TEM heat exchanger connected to the heat spreader, base plate, thermoelectric plate(s), and heat pipe/vapor chamber or solid metal thermal conductor, according to an exemplary embodiment of the present disclosure;

FIG. 9 depicts a top perspective view of the external combo liquid chiller with thermoelectric plates, fluid reservoir, pump, power controller, thermoelectric controller, and anesthetic cooling device holder, according to an exemplary embodiment of the present disclosure;

FIG. 10 depicts a top perspective of the cooling cradle plate, according to an exemplary embodiment of the present disclosure;

FIG. 11 depicts a top perspective of the anesthetic cooling device inside cooling cradle plate, thermoelectric unit, fins, and fan, according to an exemplary embodiment of the present disclosure;

FIG. 12 depicts a cross-sectional view of a solid metal cooling device sitting inside a modified cooling cradle plate, thermoelectric unit, fins, and fan, according to an exemplary embodiment of the present disclosure; and

FIG. 13 depicts a top perspective view of the housing with the external chiller, fluid reservoir, pump, power controller, and thermoelectric controller inside. Connected to and on top is the wand of the anesthetic cooling device, according to an exemplary embodiment of the present disclosure.

### DETAILED DESCRIPTION OF EMBODIMENTS

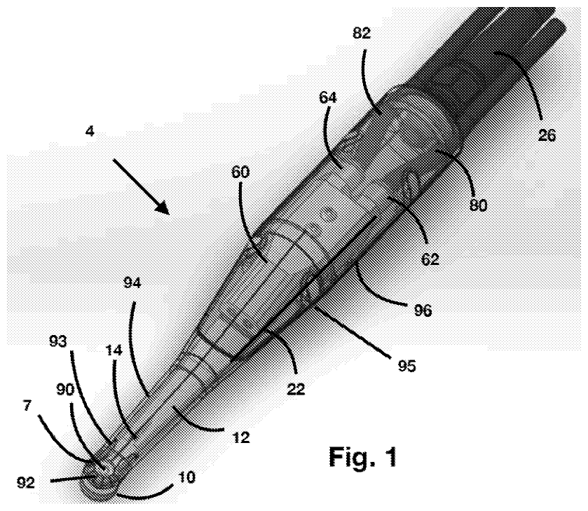

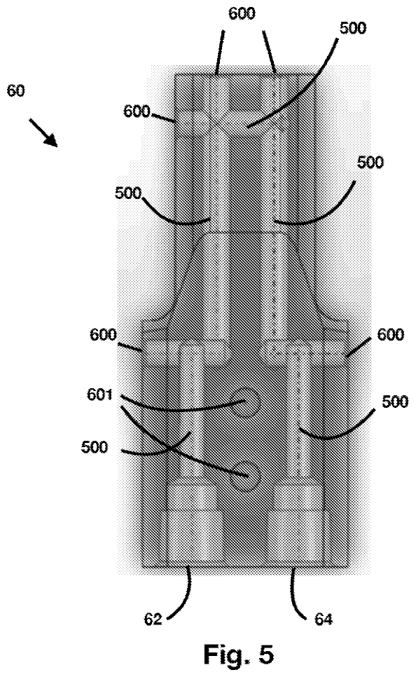

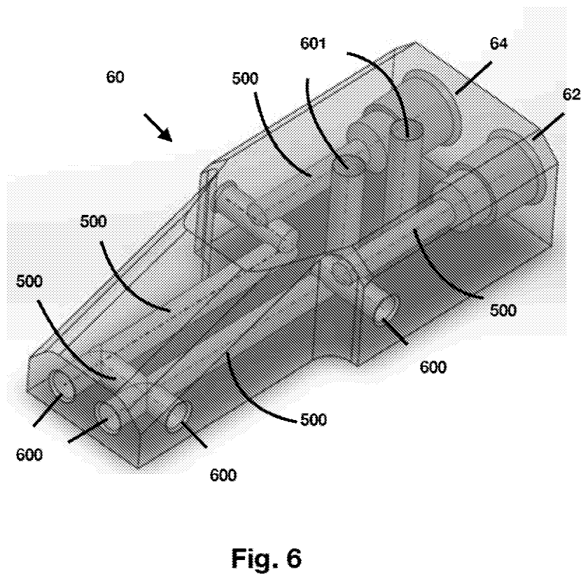

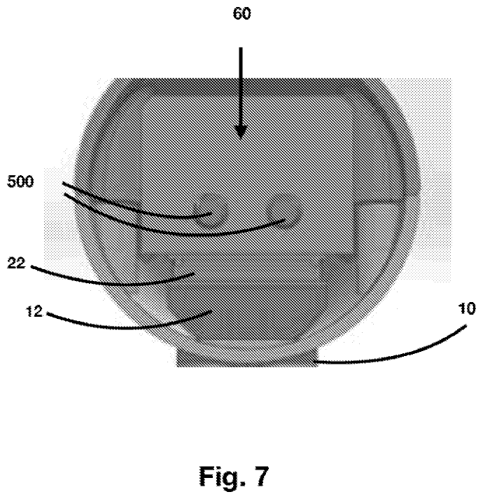

Further features and advantages of the disclosure, as well as the structure and operation of various embodiments of the disclosure, are described in detail below with reference to the accompanying FIGS. 1-14. Although the disclosure is described in the context of an intra-oral thermoelectric cooling device, the present disclosure includes devices, systems, and methods for use on any tissue.

In an embodiment of the present invention, a specialized loop coolant block is used. The anesthetic cooling device of the present invention provides transient nerve cooling block of the peripheral nervous system. The anesthetic cooling device of the present invention allows for temperature control cooling of the skin/epidermis or mucous membrane/mucosa, to alleviate pain associated with medical treatment such as injections, curettage, pulp vitality, and skin ablation applied to the human body.

The anesthetic cooling device of the present invention provides a novel and efficient heat transfer path from the device to the ambient environment. The parts and mechanism of how heat is removed from the cooling device, specifically from the thermal conductor in the handle to outside the device are disclosed herein. The present invention utilizes a novel specialized TEM heat exchanger coupled directly or indirectly through heat spreaders to the thermal conductor. The TEM heat exchanger can have one or more loops originating from the inlet and ending at the outlet.

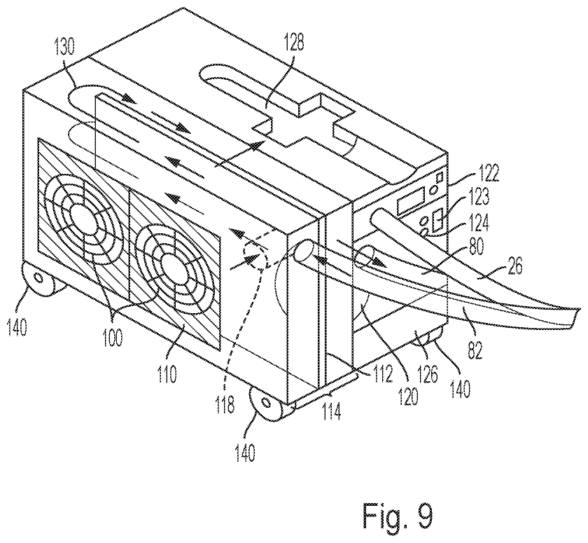

The TEM heat exchanger 60 represents the first stage of cooling conducting from external coolant chiller (as depicted in FIG. 9) contacting the hot side of thermal electric cooling modules 22. Thermal electric cooling modules are the second stage of cooling heat spreader 18, thermal conductor 12, and the device tip 10 to the target temperature.

In another exemplary embodiment, FIG. 1 depicts a top perspective view of the anesthetic cooling device 4. The anesthetic cooling device wand's tip 10 within the head 92 located on the distal end 93 of the cooling device 4 can be removable with varying sizes, shapes, and lengths and is in thermal contact with the distal end 7 of thermal conductor 12. A thermistor 14 can be coupled to the thermal conductor within the neck 94 of the wand 4. In some embodiments, the thermal conductor 12 comprises a heat pipe, a vapor chamber, and/or a solid metal thermal conductor.

A proximal gripping end 96 is used by a clinician to hold the anesthetic cooling device wand 4. The anesthetic cooling device wand 4 comprises body 95 including handle 96, distal head end 92, and proximal end 97. Distal head end 93 comprises head section 92, and a neck section 94. Distal head end 93 is sized and configured to be inserted into the mouth of a patient. In one embodiment, head section 92 comprises a thermoelectric plate that allows a user to cool a patient's tissues. In an alternative embodiment, the thermoelectric plates are in the proximal gripping end 96 and can be stacked in parallel or cascaded along the thermal conductor 12. In one embodiment, handle 96 comprises a thickness of approximately 15 mm to approximately 50 mm, and in one or more embodiments, a thickness of approximately 14 mm to approximately 40 mm. In general, dimensions for handle 96 are used that provide comfort for a user (i.e., a clinician). In one embodiment, neck section 94 and head section 92 are smaller in diameter than handle 96 and comprise a thickness of approximately 1 mm to approximately 30 mm. In one embodiment, head section 92 that interfaces with patient's tissues comprises a diameter of approximately 2 to approximately 30 mm, neck section 94 comprises a diameter of approximately 20 mm to 80 mm; and handle 96 is approximately 10 mm to approximately 30 mm wide and is approximately 100 mm to 250 mm long.

In one or more embodiments, the exterior surface of device body 95 includes one or more coatings to protect the surface and/or facilitate cleaning and/or sterilization of the cooling device 4. The device body 95, the shell surrounding the components, can be a polymer for insulation and/or a suitable thermally conductive material such as aluminum. In another embodiment, the body surrounding the TEM heat exchanger 60 and spreader 18 is a conductive metal for adjunctive heat removal.

In one embodiment, anesthetic cooling device 95 comprises an electronic assembly (not shown) located within body 95 and provides a user interface, e.g., timer buttons and timing indicator lights.

The proximal end 97 of the thermal conductor 12 is attached to cooling modules 22 in the handle 96. In some embodiments, the cooling module comprises a thermoelectric cooling module that utilizes, for example, the Peltier effect to cool certain components. Proximal cooling from the thermoelectric cooling module 22 transfer thermal energy (heat) from the tip 10 through the thermal conductor 12 to the thermoelectric module 22. The heat from the thermoelectric module 22 is transferred to the TEM heat exchanger 60.

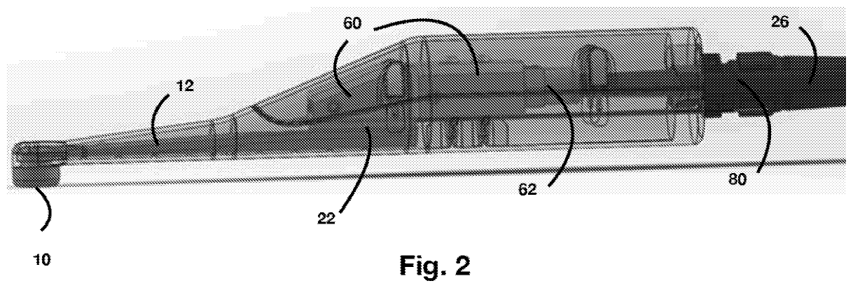

FIG. 2 depicts the wand tip 10 coupled to the thermal conductor 12. In an embodiment, the thermal conductor 12 is coupled to the thermoelectric module 22. The hot side of the thermoelectric module 22 is coupled to the TEM heat exchanger 60. The TEM heat exchanger 60 is coupled to the inlet 80 and outlet tubes 82. In some embodiments, wires connected to the thermoelectric module(s) are enclosed within tubing 26 coupled to the distal end of the wand 4.

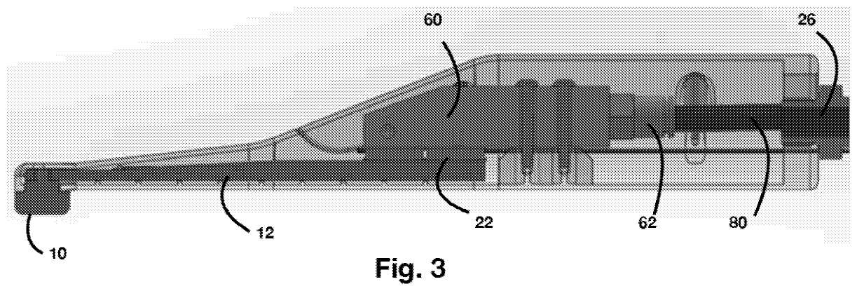

FIG. 3 depicts a thermoelectric anesthetic cooling device wand 4 with tip 10 attached to thermal conductor 12, which runs along a length of the device 4. The thermoelectric module 22 is oriented with the cold side facing the thermal conductor 12 and the hot side facing the specialized liquid loop heat exchanger 60. In some embodiments, the thermoelectric module 22 comprises a single thermoelectric plate. In other embodiments, the thermoelectric module 22 comprises a plurality of thermoelectric plates oriented in a stacked, series, or parallel configuration. The specialized TEM heat exchanger 60 is coupled to the inlet 80 and outlet tubes 82.

In some embodiments, the tip 10 is removable and interchangeable. In such an embodiment, a clinician or user can readily interchange tips 10 with different sizes and shapes for different applications. For example, in an embodiment where the wand 4 may be used to anesthetize the tissue before an injection, the tip 10 can be bulbous and concave, making it easier for the clinician to apply it to the desired area. In another embodiment where the wand 4 is used to cool a tooth and its interior during a root canal to reduce inflammation, the tip 10 would be long and conical with a pointed end, i.e., spike-like, to allow the clinician to place the tip 10 inside of the orifice of the tooth. In some embodiments, the tip 10 is interchangeable. In other embodiments, the entirety of the head section 92 (along with the tip 10) are interchangeable.

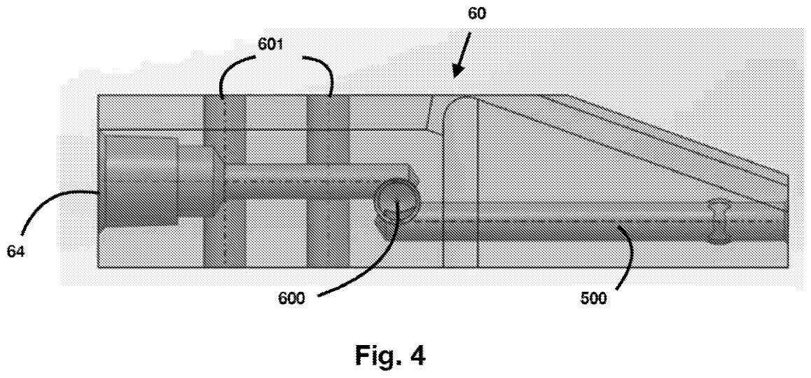

FIGS. 4-6 depict the thermoelectric module heat exchanger (or “TEM heat exchanger”) 60 in different configurations. In some embodiments, the TEM heat exchanger 60 comprises a piece of heat-conductive metal, e.g., copper or aluminum, that comprises passageways through which coolant passes. In some embodiments, the bottom of the TEM heat exchanger 60 comprises thermo-conductive block 42 that interfaces directly with of the thermoelectric module 22.

In an exemplary embodiment, the channeled loop 500 traverses the heat exchanger 60 starting from the inlet 62 and terminating at the outlet 64. In some embodiments, fasteners are used to plug the outside portions 600 enclosing the loop 500 in the heat exchanger 60. Holes 601 through the block allow for coupling the specialized TEM heat exchanger 60 to the wand 95.

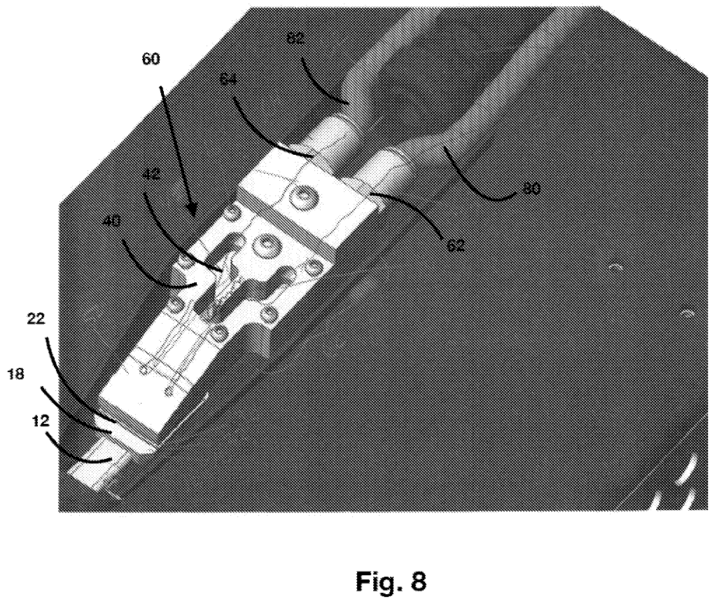

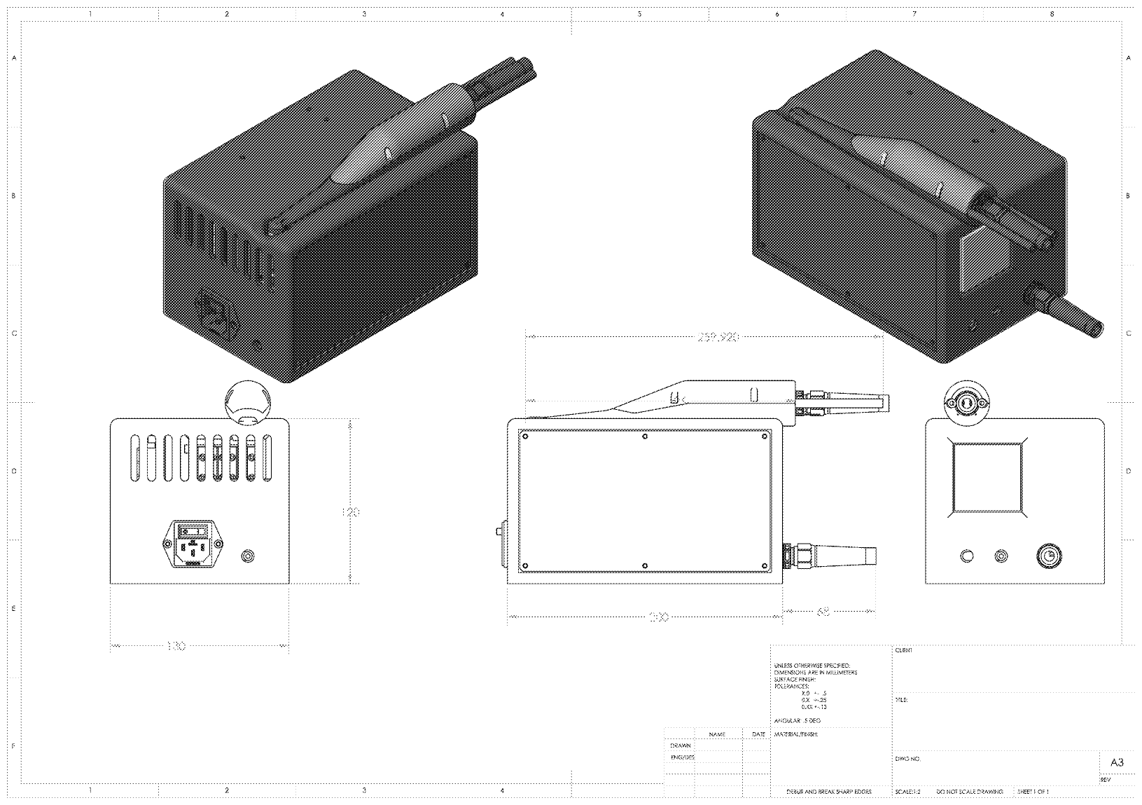

In an embodiment, a heat spreader 18 (as depicted in FIG. 8) can be disposed between the thermal conductor 12 and cold side of the thermoelectric modules 22. In another embodiment, a heat spreader 18 can be disposed in between the hot side of the thermoelectric module 22 and the TEM heat exchanger 60 to allow the TEM heat exchanger to be located posteriorly to the thermoelectric modules. In another embodiment, a plurality of thermoelectric modules 22 are stacked. In some embodiments, thermal paste or thermal pads between the thermoelectric module 22 and the heat exchanger 60 improves the heat transfer between the two surfaces. The hot side of the thermoelectric module 22 heats the heat exchanger 60, and the liquid from the inlet tube 80 into the inlet 62 absorbs the heat as it flows through the channeled loop 500, through the outlet 64 into the outlet tube 82, then into an external chiller as depicted in FIGS. 9 and 13. As a result, the TEM heat exchanger 60 transfers thermal energy from the hot side of the thermoelectric module 22 to the coolant circulating through the heat exchanger 60.

Coolant circulates through tubes 80, 82 to transfer heat from the wand of the anesthetic cooling device 4 to the external chiller 114 (FIG. 9), which then transfers the thermal energy into the ambient environment through, for example, a liquid-to-air heat exchanger. Exemplary coolants include liquid water, air, and alcohol; however, any type of coolant may be used without departing from the contemplated embodiments.

In an embodiment of the present invention, coolant is circulated through the system to cool a patient's tissue by transferring heat from the tissue to ambient air using a closed system. For example, in an exemplary embodiment, the liquid chiller 114 circulates coolant from the wand 4 through tube 82 into the reservoir 114 and is chilled by thermoelectric plates 110 with fans and fins 100. The reservoir may have a divider 112 that allows the coolant to contact the thermoelectric cooling plates 110. After being cooled by thermoelectric plates 110, the coolant is circulated to outlet 118 by pump 120, 126 and is circulated into tubing 80 towards the wand 4 of the anesthetic cooling device. The wand can reside in a cradle 128 located on cooling unit. In some embodiments, wheels 140 are used to move the unit around easily within a room. In some embodiments, the thermoelectric cooler controller 122 can be integrated with the thermoelectric module chiller, reservoir, and pump with power display 123 and timer 124. In some embodiments, the coolant chiller may also use air compressor (not shown).

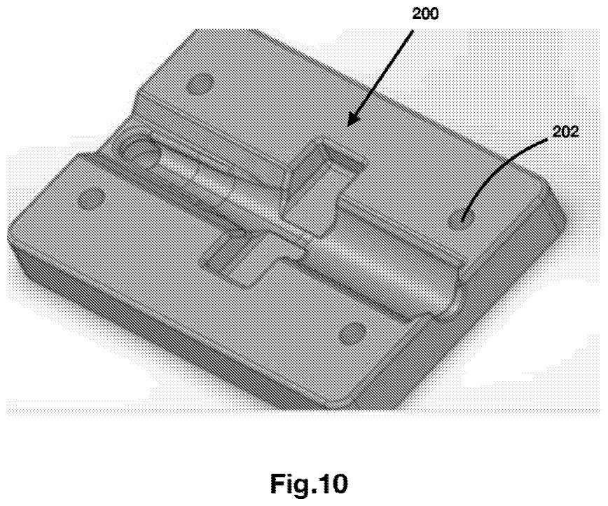

FIG. 10 depicts the cooling cradle 200 with screw holes 202. In some embodiments, the cooling cradle 200 comprises a solid metal block.

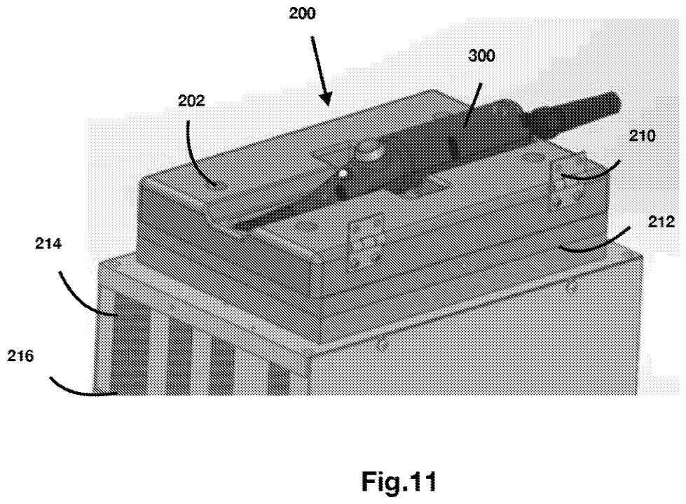

FIG. 11 depicts the cooling cradle 200 attached to the thermoelectric module 212 by way of screw holes 202. In an embodiment, the cooling cradle 200 is attached to the top of the thermoelectric module 212 that is coupled to fins 214 and/or fans 216. The wand 4 can be enclosed with a metal plate on top and on the bottom using hinges 210.

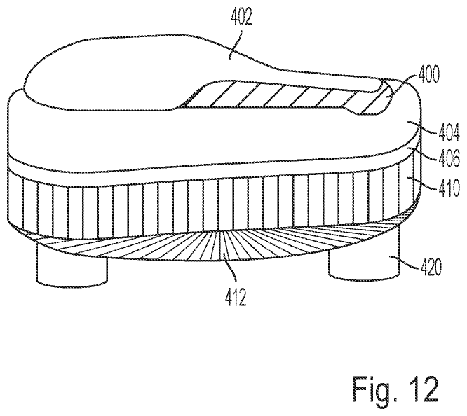

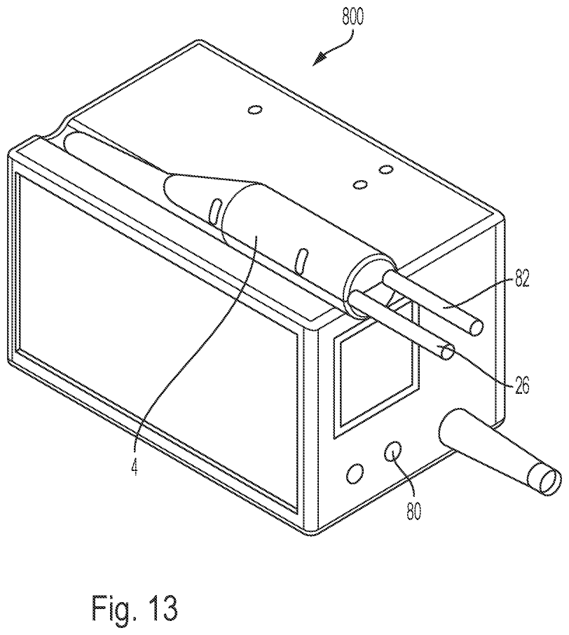

In another exemplary embodiment and with reference to FIG. 12, a solid metal wand 400 is partially covered by plastic or other insulating material 402. In the depicted embodiment, the want 400 interfaces with the cradle 200 disposed within metal plate 404, which is coupled to thermoelectric module 406 and fins 410 and/or fans 412 with four legs 420 for stability.

In another exemplary embodiment, FIG. 13 depicts operative components of the present disclosure, i.e., the external chiller, fluid reservoir, pump, power controller, and thermoelectric controller, integrated into one external unit. The wand of the anesthetic cooling device 4 is attached through tubes 80, 82 and cable 26 that can sit on top of the external box of the anesthetic cooling device. In some embodiments, a timer may be a button or switch in the cooling device, or a foot pedal that is wired or remote with the external unit.

In an embodiment of the disclosure, the methodologies and techniques described herein are implemented using the anesthetic cooling device to anesthetize tissues for injections, curettage, ablation, and for pulp testing. In an embodiment of the disclosure, the TEM heat exchanger comprises an efficient means for heat removal within the anesthetic cooling device. The disclosure has been described herein using specific embodiments for the purposes of illustration only. It will be readily apparent to one of ordinary skill in the art, however, that the principles of the disclosure can be embodied in other ways. Therefore, the disclosure should not be regarded as being limited in scope to the specific embodiments disclosed herein, but instead as being fully commensurate in scope with the following claims.

We claim:

1. An anesthetic cooling device comprising:
a wand comprising a proximal end and a distal end opposite the proximal end;
a thermoelectric module disposed within the wand having a cold side and a hot side,
a TEM heat exchanger disposed within wand and connected to the hot side of the thermoelectric module, wherein a coolant circulates into the TEM heat exchanger through a wand inlet and out of the TEM heat exchanger through a wand outlet;
a tip located at the distal end of the wand and connected to the TEM heat exchanger through a thermal conductor;
wherein thermal energy is transferred from the tip to the coolant via the thermal conductor and the TEM heat exchanger.

2. The device of claim 1 further comprising:
an external coolant chiller comprising
an inlet;
an outlet;
a pump;
a power source;
a reservoir; and
a second heat exchanger;
wherein the pump circulates coolant from the wand outlet, through the second heat exchanger, and into the TEM heat exchanger via the wand inlet; and wherein the second heat exchanger lowers the temperature of the coolant.

3. The device of claim 2, wherein the external coolant chiller further comprises a second thermoelectric module.

4. The device of claim 1 further comprising a second thermoelectric module comprising a hot side and a cold side; wherein the cold side of the second thermoelectric module is connected to the hot side of the thermoelectric module.

5. The device of claim 1, wherein the tip reaches a temperature of below 0° C.

6. The device of claim 1, wherein the tip is interchangeable.

7. The device of claim 1 further comprising a temperature sensor.

8. The device of claim 1, wherein the TEM heat exchanger is connected to the hot side of the thermoelectric module via a heat spreader.

9. A method of anesthetic cooling comprising:
contacting a patient's tissue with a tip located at the distal end of a wand;
transferring thermal energy, using a thermoelectric module, from the tip to a TEM heat exchanger, wherein the TEM heat exchanger is connected to a hot side of the thermoelectric module; and
circulating a coolant into the TEM heat exchanger through a wand inlet and out of the TEM heat exchanger through a wand outlet;
wherein the thermal energy is transferred from the tip to the TEM heat exchanger through a thermal conductor, wherein the thermal conductor is connected to the tip and to a cold side of the thermoelectric module.

10. The method of claim 8 further comprising:
circulating the coolant from the wand outlet to an inlet of an external coolant chiller;
reducing the temperature of the coolant using a second heat exchanger located within the external coolant chiller; and
circulating the coolant to the wand inlet of the TEM heat exchanger;
wherein the external coolant chiller comprises an outlet, a pump, a power source, and a reservoir.

11. The method of claim 8, wherein a second thermoelectric module is used to transfer the thermal energy from the tip to a TEM heat exchanger.

12. The method of claim 10, wherein the external coolant chiller further comprises a second thermoelectric module.

13. The method of claim 9, wherein the tip reaches a temperature of below 0° C.

14. The method of claim 8, wherein the tip is interchangeable.

15. The method of claim 9 further comprising the step of monitoring the temperature of the tip using a temperature sensor located within the wand.

16. The method of claim 8, wherein the TEM heat exchanger is connected to the hot side of the thermoelectric module via a heat spreader.
